(19) United States
(12) Patent Application Publication
(10) Pub. No.: US 2003/0187055 A1
(43) Pub. Date: Oct. 2, 2003

(54) Synergistic pharmaceutical combinations for treating obesity

(76) Inventors: Donald K. Riker, Chattanooga, TN (US); Michael Y. Law, Chattanooga, TN (US)

Correspondence Address: Douglas T. Johnson, Miller & Martin LLP, 1000 Volunteer Building, 832 Georgia Avenue, Chattanooga, TN, 37402, US

(21) Appl. No.: 10/373,726

(22) Filed: Feb. 25, 2003

Related U.S. Application Data
(60) Provisional application No. 60/359,919, filed on Feb. 25, 2002.

(51) Int. Cl. A61K 38/04; A61K 31/405

(52) U.S. Cl. 514/419; 514/19

(57) ABSTRACT

This invention relates to a novel composition which contains 5-hydroxytryptophan (5-HTP) with or without Vitamin B6 (pyridoxal phosphate) as a cofactor in combination with (−)hydroxycitric acid (HCA). This composition may be used for the treatment of obesity or appetite suppression.

### RELATED APPLICATION

This application claims priority to provisional application Ser. No. 60/359,919 filed Feb. 25, 2002.

### BACKGROUND OF THE INVENTION

1. Field of the Invention

This invention relates to a novel composition which contains 5-hydroxytryptophan (5-HTP) with or without Vitamin B6 (pyridoxal phosphate) as a cofactor in combination with (−)hydroxycitric acid (HCA) and a method for the treatment of obesity and appetite suppression.

2. Description of Related Art

Studies have documented that substances which increase brain serotonin (5-hydroxytryptamine, 5-HT), such as fenfluramine, are effective anorectic agents to help obese patients lose weight and to decrease cravings for sweets and carbohydrates. 5-Hydroxytryptophan, abbreviated 5-HTP, is the immediate precursor of 5-HT. An effect of administering 5-HTP is to increase brain 5-HT. Previous studies in animals and humans have established that oral administration of 5-HTP increases brain 5-HT. It is desirable to have such an anorectic agent for human therapy which does not present the well-known unwanted and often dangerous effects typical of amphetamine and fenfluramine or their congeners, ranging from nausea and insomnia to hypertension and cardiac arrhythmias.

Substances which increase brain serotonin (5-HT) act to decrease hunger and cravings for food, especially for carbohydrates and sweets. The biosynthesis of 5-HT in the brain proceeds from the uptake of dietary tryptophan by the neuron, followed by its conversion to 5-hydroxytryptophan (5-HTP) by the enzyme tryptophan hydroxlyase. The main metabolic pathway to 5-HT is via decarboxylation of 5-HTP by the enzyme L-aromatic amino acid decarboxylase. Like other amino acid decarboxylases, this enzyme needs Vitamin B6 (pyridoxal phosphate) as a cofactor.

5-HTP is the direct precursor of serotonin. 5-HTP is a natural compound isolated from the seeds of an African plant called Griffonia simplicifolia, grown mostly in Ghana and the Ivory Coast. It can also be made synthetically in the laboratory. Its toxicity is extremely low as noted by lethal dose (LD) studies. Studies conducted in the rat and mouse have demonstrated that the LD.sub.50 is negligible as compared to therapeutic doses: LD50 per os in the mouse 2500 mg/kg; LD50 i.p. 1400 mg/kg. However, synthetically produced 5-HTP has been associated with a cluster of symptoms called eosinophilia-myalgia syndrome (EMS). EMS is a serious systemic illness characterized by elevations of certain white blood cells and severe muscle pain. In 1989 there was an epidemic outbreak of EMS, triggered by the consumption of synthetically produced L-tryptophan produced by a fermentation process. More than 1,500 cases including at least 37 deaths were reported to the CDC as of February, 2001. 5-HTP isolated from the seeds of Griffonia simplicifolia does not require the use of a fermentation process and is a safer product.

(−)Hydroxycitric acid (abbreviated herein as HCA) is a naturally-occurring substance found chiefly in species of Garcinia, such as Garcinia cambogia. Several synthetic derivatives of citric acid have been investigated extensively in regard to their ability to inhibit the production of fatty acids from carbohydrates, to suppress appetite, and to inhibit weight gain. (Sullivan, A. C., et al., American Journal of Clinical Nutrition 1977; 30:767.) Weight loss benefits were first ascribed to HCA, its salts and its lactone in U.S. Pat. No. 3,764,692 granted to John M. Lowenstein in 1973. The claimed mechanisms of action for HCA have been summarized in at least two United States Patents. In U.S. Pat. No. 5,626,849 these mechanisms are described as follows: “(−) HCA reduces the conversion of carbohydrate calories into fats. It does this by inhibiting the actions of ATP-citrate lyase, the enzyme which converts citrate into fatty acids and cholesterol in the primary pathway of fat synthesis in the body. The actions of (−) HCA increase the production and storage of glycogen (which is found in the liver, small intestine and muscles of mammals) while reducing both appetite and weight gain. (−) Hydroxycitric acid also causes calories to be burned in an energy cycle similar to thermogenesis. HCA also increases the clearance of LDL cholesterol.” U.S. Pat. No. 5,783,603 teaches that HCA serves to disinhibit the metabolic breakdown and oxidation of stored fat for fuel via its effects upon the compound malonyl CoA and that gluconeogenesis takes place as a result of this action. The position that HCA acts to unleash fatty acid oxidation by negating the effects of malonyl CoA with gluconeogenesis as a consequence (McCarty M. F., Medical Hypotheses 1994; 42:215-225) is maintained in U.S. Pat. No. 5,914,326.

There is evidence from animal studies that ingested HCA will lower blood lipids levels, and it is known that high levels of circulating free fatty acids are often related to insulin resistance and thereby to erratic blood glucose control. However, this is an indirect effect rather than a direct one; it is analogous to maintaining—accurately—that substantial weight loss improves insulin resistance in many individuals.

### SUMMARY OF THE INVENTION

The present invention recognizes and addresses the foregoing disadvantages, and others, of prior art compositions and methods.

Briefly, the present invention is a novel composition for the treatment of obesity and appetite suppression. The applicants have discovered that administering 5-hydroxytryptophan (5-HTP) in combination with HCA to an individual results in unexpected potent anorectic activity that far exceeds the expected anorectic activity of combining 5-HTP and HCA. Additionally, the present invention provides an effective method for treating obesity and suppressing the appetite using naturally substances with little or no side effects.

One novel aspect of the invention is the use of 5-HTP and HCA for appetite suppression and promoting weight and fat loss.

A second novel aspect of the invention relates to the synergistic effect of using 5-HTP and HCA to control weight loss. In particular, capsules containing 5-HTP (25 mg) and HCA (250 mg) orally administered one to three times per day are particularly effective.

Accordingly, it is an object of the present invention to provide an appetite suppressant product and method of use which provides unexpected and surprisingly potent anorectic activity.

More particularly, it is an object of the present invention to provide appetite suppressant products having little or no adverse side effects.

More particularly, it is an object of the present invention to provide appetite suppressant products which consist of naturally occurring substances, naturally derived 5-HTP and HCA.

Moreover, it is an object of the present invention to provide a method for the treatment of obesity, appetite suppression, and the promotion of weight and fat loss.

Additional objects and advantages of the invention will be set forth in part in the following description, or may be obvious from the description, or may be learned through practice of the invention.

### DETAILED DESCRIPTION OF THE INVENTION

The compounds according to the present invention are found in two different classes of therapeutically active substances, i.e. one class comprising the precursor of the neurotransmitter serotonin, such as commercially available 5-hydroxytryptophan and its salts. It should be understood that any of its L, D or racemic forms are suitable, however, precursors are preferably in L form. The second class comprises HCA or derivatives thereof (wherein “derivatives thereof” also encompasses natural products and their extracts containing same) that serve as metabolic blocking drugs. Preferred hydroxycitric acid derivatives are the salts and esters thereof and the natural products and their extracts containing same, as specified below. It should be understood that whenever in the present specification reference is made for the sake of simplicity to “HCA”, the naturally occurring compound, i.e (−)hydroxycitric acid, is meant. Hydroxycitric acid and derivatives thereof may occur as extracts of natural products containing hydroxycitirc acid at high concentrations, such as the extract of the fruits of Garcinia (Garcinia Cambodia, Garcinia astroviridis, Garcinia indica, Garcinia citrin), of the fruits of Malabar Tamarind or Gorikapuli (Lewis Y. L., Neelakantan S., Phyto-chemistry 1965; 4:619), (Streenivasan A., Vankataraman R., Current Science 1959; 4:151) or other extract of natural products containing same.

The term “therapeutically active substance” as used herein is intended to mean any physiologically or pharmacologically active substance that produces a localized or systemic effect in animals, in particular in mammals, including humans, primates and domestic animals.

It has been found that it is preferable to administer to an individual in need thereof in a single or multiple regimen dose of at least 0.5 mg/day of 5-hydroxytryptophan and or an equivalent amount of a pharmacologically acceptable salt thereof. The free acid form and various salts of (−)-hydroxycitric acid (calcium, magnesium, potassium and sodium) have been available commercially for several years. Any of these materials can be used to fulfill the invention revealed here. Exact dosing will depend upon the form of HCA used, the weight of the individual involved, and the other components of the diet.

This invention provides a composition providing between about 0.5 mg to 5 g of 5-HTP, between about 0.5 mg to 10 g of HCA, and between about 0.05 mg to 2 g of Vitamin B6 for administration up to 3 times daily. Because Vitamin B6 doses above 2 g per day may be toxic, high doses of Vitamin B6 are not recommended.

In the preferred embodiment, the ratio of 5-HTP to HCA is less than 1:5 by weight, and preferably about 1:10 by weight. The preferred embodiment is a tablet containing 25 mg of naturally derived 5-HTP, 2 mg of Vitamin B6 as pyridoxine hydrochloride, and 250 mg of HCA. The tablet may be taken up to 3 times daily.

Additional ingredients may be added to the composition, including, but not limited to vitamins, minerals and other trace elements. These supplements can be varied as desired but are typically equal to the RDA or greater based on 2,000 calories. A variety of herbal supplements may also be added to the composition of the present invention including ginseng root and others.

In a pilot clinical study, Garcinia cambogia (with a concentration of 50% HCA) and Griffonia simplicifolia (with a concentration of about 95% 5-HTP) were given to 12 human subjects three times daily for four weeks. The amount of Garcinia cambogia was kept constant at 500 mg per dose. Group A received Griffonia simplifolia standardized to 25 mg 5-HTP and Group B received Griffonia simplifolia standardized to 50 mg 5-HTP. The weights, body fat percentages, body mass index (BMI), waist measurements, visual analog scales for hunger, fullness, and food cravings, general well-being questionnaires, and vital signs were recorded on all subjects over 4 weeks. Group A lost more weight and body fat than Group B. Group A also showed more of a decrease of hunger and food cravings and an increase of fullness. This trend shows the effectiveness of 25 mg of 5-HTP and 500 mg of Garcinia cambogia.

The only reported side effect of this combination is nausea and diarrhea which is mild in severity. However, it has been found that these complaints are temporary and usually disappear. The vital signs of the participants did not change significantly during the course of the study. Another common complaint was loss of appetite.

The composition according to the present invention can be formulated for administration by any suitable route such as the oral, rectal, nasal, or parenteral administration route. Thus, the composition may be in the form of tablets, capsules, suspensions, emulsions, solutions, injectables, suppositories, sprays, aerosols or another suitable form.

Formulations for oral use include tablets, which contain the active ingredients in admixture with non-toxic pharmaceutically acceptable excipients. These excipients may be, for example, inert diluents, such as calcium carbonate, sodium chloride, lactose, calcium phosphate or sodium phosphate; granulating and disintegrating agents, for example, potato starch or alginic acid; binding agents, for example, starch, gelatin or acacia; and lubricating agents, for example, magnesium stearate, stearic acid or talc. Other pharmaceutically acceptable excipients can be colorants, flavouring agents, plasticizers, humectants etc. The tablets may be uncoated or they may be coated by known techniques, optionally to delay disintegration and absorption in the gastrointestinal tract and thereby provide a sustained action over a longer period. For example, a time delay material such as glyceryl monostearate or glyceryl distearate may be employed. Formulations for oral use may also be presented as chewing tablets, or as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin, or olive oil. Powders, dispersible powders or granules suitable for preparation of an aqueous suspension by addition of water are also convenient dosage forms of the present invention.

Formulations for oral use also include buccal/oral strips, which contain the active ingredients in portable, rapidly-dissolving complex carbohydrate or starch-based strips. These strips consist of active ingredients, flavorings, colors or excipients that rapidly dissolve in the buccal-oral cavity. These strips are portable and small and do not require swallowing a pill or liquid. Alternatively, the strips may be dissolved directly into food or drinks.

Formulation as a suspension provides the active ingredients in admixture with a dispersing or wetting agent, suspending agent and one or more preservatives. Suitable dispersing or wetting agents are, for example, naturally-occurring phosphatides, as e.g. lecithin, or condensation products of ethylene oxide with e.g. a fatty acid, a long chain aliphatic alcohol or a partial ester derived from fatty acids and a hexitol or a hexitol anhydrides, for example, polyoxyethylene stearate, polyoxyethylene sorbitol monooleate, polyoxyethylene sorbitan monooleate etc. Suitable suspending agents are, for example, sodium carboxymethylcellulose, methylcellulose, sodium alginate etc. The formulation may also be administered parenterally (intravenous, intramuscular, subcutaneous or the like) in dosage forms or formulations containing conventional, non-toxic pharmaceutically acceptable carriers and adjuvants.

Preferably, the composition of the present invention comprises a combination product containing naturally derived 5-hydroxytryptophan with or without Vitamin B6 (pyridoxal phosphate) as a cofactor in combination with the HCA salts, i.e. in the case of a tablet; one tablet comprises a mixture of the three active components. However, the composition of the present invention may also be presented in one package comprising two separate containers, one container comprising dosage form of the 5-hydroxytryptophan/Vitamin B6 and the other container comprising a dosage form of the HCA. In such cases, special instructions for substantially concomitant use of the two drugs should be enclosed with the product. The two dosage forms can be the same or they may be different, preferably the two dosage forms are the same.

It is to be understood that according to the teachings of the invention, the invention can be practiced by administering serotonin-mediated neurotransmission stimulating compounds, for example, 5-hydroxytryptophan, to a subject as a single dose one or more times per day, or as a plurality of unit doses one or more times per day without deviating from the teachings of the invention.

It is to be further understood that the present invention also includes a method of making a composition for the treatment of obesity or appetite suppression, wherein the method comprises a step of mixing a serotonin precursor such as 5-hydroxytryptophan with HCA.

In one aspect the present invention relates to a method for treatment of overweight or obese individuals, in particular in humans, or for reducing the adipose tissue mass/lean mass body mass ratio of an individual, in particular a human.

In the present context the term “overweight” is used as an indication of a body with a weight exceeding the “desirable weight”, whereas the term “obesity” is used when the body weight is 20% or more above the “desirable weight.” Desirable weights for humans are defined as a body mass index less than or equal to 24.

In another aspect, the invention can be used along with other appetite suppressant drugs, such as N,N-dimethyl-1-[1-(4-chlorophenyl)cyclobutyl]-3-methylbutylamine hydrochloride monohydrate (sibutramine hydrochloride), synephrine, amphetamine, phentermine, diethylpropion, and phendimetrazine or similarly-acting agents to increase the weight loss which would occur with the use of these agents alone.

In addition, the invention can be used with other drugs that effect fat uptake to enhance the effect of the invention, for example, lipase inhibiting drugs such as Orlistat, and Xenical, or lipid absorbing polysaccharides such as Chitosan, or alpha amylase inhibitors such as acarbose, voglibose, miglitol, emiglitate, camiglibose, salbostatin that reduce starch intake in humans. However, the composition of the present invention does not require the use of drugs that effect fat uptake or active ingredients such as phenylpropanolamine, ephedrine, ephedra alkaloids, ma huang (Chinese ephedra) or citrus aurantium (bitter orange peel).

In a further aspect, the present invention also relates to the use of a combination of 5-hydroxytryptophan, Vitamin B6 (pyridoxal phosphate) as an optional cofactor, and HCA for the manufacture of a composition for appetite suppression, the treatment of obesity or diseases aggravated thereof.

The invention is further illustrated by means of the following illustrative embodiment, which is given for the purpose of illustration only and is not meant to limit the invention to the particular components and amounts disclosed. The following example shows the preferred embodiment for producing appetite suppressant products and products which promote weight and fat loss, comprising 5-hydroxytryptophan with or without Vitamin B6 (pyridoxal phosphate) as a cofactor in combination with HCA.

One coated tablet of the composition according to the present invention could have the following ingredients:

| | | % by |
|---|---|---|
| Ingredient | Mg/caplet | Weight |
| Active Ingredients: | | |
| Vitamin B6 (pyridoxine hydrochloride) | 26.000 | 2.989% |
| Garcinia cambogia extract | 500.000 | 57.471% |
| (50% HCA) | | |
| Panax ginseng root (4% ginsenosides) from 80% | 5.000 | 0.575% |
| Marker: Ginsenosides | | |
| | (4.000) | |
| 5-Hydroxytryptophan (from 95%) | 26.500 | 3.046% |
| Inactive Ingredients: | | |
| Dicalcium phosphate | 70.500 | 8. 103% |
| Microcrystalline cellulose | 170.000 | 19.540% |
| Croscarmellose sodium | 36.000 | 4.137% |
| Stearic acid | 24.000 | 2.759% |
| Silicon dioxide | 6.000 | 0.690 |
| Magnesium stearate | 6.000 | 0.690% |
| Total Weight of core tablet: | 870.000 | 100.000% |
| Dri-Klear Clear 010 (53-859010-00) HPMC | 13.000 | |
| Powder | | |
| Total Weight of coated tablet: | 883.000 | |

We claim:

1. A composition comprising an effective amount of:
(a) a compound which enhances serotonin-mediated neurotransmission, or a pharmaceutically acceptable salt thereof, wherein said compound is selected from the group consisting of precursors of serotonin, pro-drugs of serotonin, or an intermediate in the biosynthesis of serotonin; and
(b) a compound selected from the group consisting of (−)-hydroxycitric acid, a pharmaceutically acceptable salt thereof or (−)-hydroxycitric acid lactone.

2. The composition according to claim 1, wherein the compound which enhances serotonin-mediated neurotransmission, or a pharmaceutically acceptable salt thereof is 5-hydroxytryptophan.

3. The composition according to claim 1, wherein the compound which enhances serotonin-mediated neurotransmission, or a pharmaceutically acceptable salt thereof is naturally derived 5-hydroxytryptophan.

4. The composition according to claim 1, wherein the compound which enhances serotonin-mediated neurotransmission, or a pharmaceutically acceptable salt thereof is selected from the group consisting of L-tryptophan, L-5-hydroxytryptophan, diethyl N-benzyloxycarbonyl-5-benzyloxycarbonyloxy-L-tryptophyl-L-aspartate, dibenzyl N-benzyloxycarbonyl-5-hydroxy-L-tryptophanylaspartate, 5-Hydroxy-L-tryptophyl-L-aspartic acid trihydrate, diethyl N-benzyloxycarbonyl-5-hydroxy-L-tryptophyl-L-glutamate, diethyl 5-hydroxy-L-tryptophyl-L-glutamate hydrochloride, dibenzyl L-benzyloxycarbonyl-5-hydroxytryptophyl-L-glutamate, 5-hydroxy-L-tryptophyl-L-glutamic acid, pentachlorophenyl ester of N-benzyloxycarbonyl-5-hydroxy-L-tryptophan, methyl ester of N-benzyloxycarbonyl-5-hydroxy-L-tryptophyl-L-tyrosine, N-Acetyl-5-hydroxy-L-tryptophan, methyl ester of N-acetyl-5-hydroxy-L-tryptophyl-L-tyrosine, methyl ester of n-acetyl-5-hydroxy-L-tryptophyl-5-hydroxy-L-tryptophan, 5-hydroxy-L-tryptophyl-L-alaninc hydrate, 5-hydroxy-L-tryptophan-L-valine, 5-hydroxy-L-tryptophyl-L-leucine, 5-hydroxy-L-tryptophyl-L-proline, 5-hydroxy-L-tryptophyl-L-phenylalanine, 5-hydroxy-L-tryptophyl-5-hydroxy-L-tryptophan, 5-hydroxy-L-tryptophyl-L-tryptophan, 1-5-hydroxytryptophyl-L-serine, 5-hydroxy-L-tryptophyl-L-arginine, 5-hydroxy-L-tryptophylglycine, 5-hydroxy 1-tryptophyl-gamma-aminobutyric acid, 5-hydroxy-L-tryptophanamide hydrate, methyl ester of 5-hydroxy-L-tryptophyl-L-histidine, benzyl ester of L-5-hydroxytryptophan, benzyl ester of N-benzyloxycarbonyl-5-hydroxy-L-tryptophyl-5-hydroxy-L-tryptophan, 5-Hydroxy-L-tryptophyl-5-hydroxy-L-tryptophan hemihydrate, 5-hydroxytryptophan inosinate, theophylline salt of (DL) 5-hydroxytryptophan, and combinations thereof.

5. The composition according to claim 1, comprising between about 0.5 mg to about 5 g of 5-hydroxytryptophan.

6. The composition according to claim 1, comprising between about 0.5% to about 15% by weight 5-hydroxytryptophan.

7. The composition according to claim 1, comprising between about 0.5 mg to 10 g by weight (−)-hydroxycitric acid.

8. The composition according to claim 1, comprising between about 0.5% to about 30% by weight (−)-hydroxycitric acid.

9. The composition according to claim 1, further comprising an effective amount of Vitamin B6.

10. The composition according to claim 9, comprising between about 0.05 mg to 2 g of Vitamin B6.

11. The composition according to claim 1, further comprising an effective amount of other anorectic agents selected from the group consisting of phentermine, diethylpropion, and phendimetrazine.

12. A composition comprising:
(a) At least 0.5 mg of 5-hydroxytryptophan, and
(b) At least 0.5 mg of (−)-hydroxycitric acid or any derivative thereof.

13. A method for the treatment of obesity or inducing weight loss in human subjects comprising administration of a composition comprising:
(a) a compound which enhances serotonin-mediated neurotransmission, or a pharmaceutically acceptable salt thereof, wherein said compound is selected from the group consisting of precursors of serotonin, pro-drugs of serotonin, or an intermediate in the biosynthesis of serotonin; and
(b) a compound selected from the group consisting of (−)-hydroxycitric acid, a pharmaceutically acceptable salt thereof or (−)-hydroxycitric acid lactone.

14. The method according to claim 13, wherein the compound which enhances serotonin-mediated neurotransmission, or a pharmaceutically acceptable salt thereof is 5-hydroxytryptophan.

15. The method according to claim 13, wherein the compound which enhances serotonin-mediated neurotransmission, or a pharmaceutically acceptable salt thereof is naturally derived 5-hydroxytryptophan.

16. The method according to claim 13, wherein compound which enhances serotonin-mediated neurotransmission, or a pharmaceutically acceptable salt thereof is selected from the group consisting of L-tryptophan, L-5-hydroxytryptophan, diethyl N-benzyloxycarbonyl-5-benzyloxycarbonyloxy-L-tryptophyl-L-aspartate, dibenzyl N-benzyloxycarbonyl-5-hydroxy-L-tryptophanylaspartate, 5-Hydroxy-L-tryptophyl-L-aspartic acid trihydrate, diethyl N-benzyloxycarbonyl-5-hydroxy-L-tryptophyl-L-glutamate, diethyl 5-hydroxy-L-tryptophyl-L-glutamate hydrochloride, dibenzyl L-benzyloxycarbonyl-5-hydroxytryptophyl-L-glutamate, 5-hydroxy-L-tryptophyl-L-glutamic acid, pentachlorophenyl ester of N-benzyloxycarbonyl-5-hydroxy-L-tryptophan, methyl ester of N-benzyloxycarbonyl-5-hydroxyl-L-tryptophy-L-tyrosine, N-Acetyl-5-hydroxy-L-tryptophan, methyl ester of N-acetyl-5-hydroxy-L-tryptophyl-L-tyrosine, methyl ester of n-acetyl-5-hydroxy-L-tryptophyl-5-hydroxy-L-tryptophan, 5-hydroxy-L-tryptophyl-L-alaninc hydrate, 5-hydroxy-L-tryptophan-L-valine, 5-hydroxy-L-tryptophyl-L-leucine, 5-hydroxy-L-tryptophyl-L-proline, 5-hydroxy-L-tryptophyl-L-phenylalanine, 5-hydroxy-L-tryptophyl-5-hydroxy-L-tryptophan, 5-hydroxy-L-tryptophyl-L-tryptophan, 1-5-hydroxytryptophyl-L-serine, 5-hydroxy-L-tryptophyl-L-arginine, 5-hydroxy-L-tryptophylglycine, 5-hydroxy 1-tryptophyl-gamma-aminobutyric acid, 5-hydroxy-L-tryptophanamide hydrate, methyl ester of 5-hydroxy-L-tryptophyl-L-histidine, benzyl ester of L-5-hydroxytryptophan, benzyl ester of N-benzyloxycarbonyl-5-hydroxy-L-tryptophyl-5-hydroxy-L-tryptophan, 5-Hydroxy-L-tryptophyl-5-hydroxy-L-tryptophan hemihydrate, 5-hydroxytryptophan inosinate, theophylline salt of (DL) 5-hydroxytryptophan, and combinations thereof.

17. The composition according to claim 13, comprising between about 0.5 mg to about 5 g of 5-hydroxytryptophan.

18. The composition according to claim 13, comprising between about 0.5% to about 15% by weight 5-hyydroxytryptophan.

19. The method according to claim 13, comprising between about 0.5 mg to 10 g of (−)-hydroxycitric acid.

20. The method according to claim 13, comprising between about 0.5% to about 30% by weight of (−)-hydroxycitric acid.

21. The method according to claim 13, wherein the composition further comprises an effective amount of Vitamin B6.

22. The method according to claim 13, further comprising administration of an effective amount of other anorectic agents selected from the group consisting of phentermine diethylpropion, and phendimetrazine.

23. The method according to claim 13, wherein the compound which enhances serotonin-mediated neurotransmission, or a pharmaceutically acceptable salt thereof is administered at a dose ranging from about 0.5 mg/day to about 5 g/day and a compound selected from the group consisting of (−)-hydroxycitric acid, a pharmaceutically acceptable salt thereof or (−)-hydroxycitric acid lactone is administered at a dose ranging from 0.5 mg to 10 g per day.

24. A method for suppressing the appetite of human subjects comprising administration of a composition comprising:
(a) a compound which enhances serotonin-mediated neurotransmission, or a pharmaceutically acceptable salt thereof, wherein said compound is selected from the group consisting of precursors of serotonin, pro-drugs of serotonin, or an intermediate in the biosynthesis of serotonin; and
(b) a compound selected from the group consisting of (−)-hydroxycitric acid, a pharmaceutically acceptable salt thereof or (−)-hydroxycitric acid lactone.

25. The method according to claim 24, wherein the compound which enhances serotonin-mediated neurotransmission, or a pharmaceutically acceptable salt thereof is 5-hydroxytryptophan.

26. The method according to claim 24, wherein the compound which enhances serotonin-mediated neurotransmission, or a pharmaceutically acceptable salt thereof is naturally derived 5-hydroxytryptophan.

27. The method according to claim 24, wherein compound which enhances serotonin-mediated neurotransmission, or a pharmaceutically acceptable salt thereof is selected from the group consisting of L-tryptophan, L-5-hydroxytryptophan, diethyl N-benzyloxycarbonyl-5-benzyloxycarbonyloxy-L-tryptophyl-L-aspartate, dibenzyl N-benzyloxycarbonyl-5-hydroxy-L-tryptophanylaspartate, 5-Hydroxy-L-tryptophyl-L-aspartic acid trihydrate, diethyl N-benzyloxycarbonyl-5-hydroxy-L-tryptophyl-L-glutamate, diethyl 5-hydroxy-L-tryptophyl-L-glutamate hydrochloride, dibenzyl L-benzyloxycarbonyl-5-hydroxytryptophyl-L-glutamate, 5-hydroxy-L-tryptophyl-L-glutamic acid, pentachlorophenyl ester of N-benzyloxycarbonyl-5-hydroxy-L-tryptophan, methyl ester of N-benzyloxycarbonyl-5-hydroxyl-L-tryptophy-L-tyrosine, N-Acetyl-5-hydroxy-L-tryptophan, methyl ester of N-acetyl-5-hydroxy-L-tryptophyl-L-tyrosine, methyl ester of n-acetyl-5-hydroxy-L-tryptophyl-5-hydroxy-L-tryptophan, 5-hydroxy-L-tryptophyl-L-alaninc hydrate, 5-hydroxy-L-tryptophan-L-valine, 5-hydroxy-L-tryptophyl-L-leucine, 5-hydroxy-L-tryptophyl-L-proline, 5-hydroxy-L-tryptophyl-L-phenylalanine, 5-hydroxy-L-tryptophyl-5-hydroxy-L-tryptophan, 5-hydroxy-L-tryptophyl-L-tryptophan, 1-5-hydroxytryptophyl-L-serine, 5-hydroxy-L-tryptophyl-L-arginine, 5-hydroxy-L-tryptophylglycine, 5-hydroxy 1-tryptophyl-gamma-aminobutyric acid, 5-hydroxy-L-tryptophanamide hydrate, methyl ester of 5-hydroxy-L-tryptophyl-L-histidine, benzyl ester of L-5-hydroxytryptophan, benzyl ester of N-benzyloxycarbonyl-5-hydroxy-L-tryptophyl-5-hydroxy-L-tryptophan, 5-Hydroxy-L-tryptophyl-5-hydroxy-L-tryptophan hemihydrate, 5-hydroxytryptophan inosinate, theophylline salt of (DL) 5-hydroxytryptophan, and combinations thereof.

28. The composition according to claim 24, comprising between about 0.5 mg to about 5 g of 5-hydroxytryptophan.

29. The composition according to claim 24, comprising between about 0.5% to about 15% by weight 5-hyydroxytryptophan.

30. The method according to claim 24, comprising between about 0.5 mg to 10 g of (−)-hydroxycitric acid.

31. The method according to claim 24, comprising between about 0.5% to about 30% by weight (−)-hydroxycitric acid.

32. The method according to claim 24, wherein the composition further comprises an effective amount of Vitamin B6.

33. The method according to claim 24, further comprising administration of an effective amount of other anorectic agents selected from the group consisting of phentermine diethylpropion, and phendimetrazine.

34. The method according to claim 24, wherein the compound which enhances serotonin-mediated neurotransmission, or a pharmaceutically acceptable salt thereof is administered at a dose ranging from about 0.5 mg/day to about 5 g/day and a compound selected from the group consisting of (−)-hydroxycitric acid, a pharmaceutically acceptable salt thereof or (−)-hydroxycitric acid lactone is administered at a dose ranging from 0.5 mg to 10 g per day.

35. A method for the reduction of body mass of human subjects comprising administration of a composition comprising:
(a) a compound which enhances serotonin-mediated neurotransmission, or a pharmaceutically acceptable salt thereof, wherein said compound is selected from the group consisting of precursors of serotonin, pro-drugs of serotonin, or an intermediate in the biosynthesis of serotonin; and
(b) a compound selected from the group consisting of (−)-hydroxycitric acid, a pharmaceutically acceptable salt thereof or (−)-hydroxycitric acid lactone.

36. A method for the treatment of stress comprising administration of a composition comprising:
(a) a compound which enhances serotonin-mediated neurotransmission, or a pharmaceutically acceptable salt thereof, wherein said compound is selected from the group consisting of precursors of serotonin, pro-drugs of serotonin, or an intermediate in the biosynthesis of serotonin; and
(b) a compound selected from the group consisting of (−)-hydroxycitric acid, a pharmaceutically acceptable salt thereof or (−)-hydroxycitric acid lactone.

37. A composition comprising:
(c) between about 0.5% to 15% by weight of a compound which enhances serotonin-mediated neurotransmission, or a pharmaceutically acceptable salt thereof, wherein said compound is selected from the group consisting of precursors of serotonin, pro-drugs of serotonin, or an intermediate in the biosynthesis of serotonin; and
(d) between about 0.5% to 30% by weight of a compound selected from the group consisting of (−)-hydroxycitric acid or derivatives thereof.

37. The composition according to claim 1, comprising 5-hydroxytryptophan and (−)-hydroxycitric acid in a ratio of 1 to 10 by weight.

38. The composition according to claim 1, comprising 5-hydroxytryptophan and (−)-hydroxycitric acid in a ratio of less than 1:5 by weight.
